(19) United States
(12) Patent Application Publication
(10) Pub. No.: US 2015/0150922 A1
(43) Pub. Date: Jun. 4, 2015

(54) COMPOSITION FOR USE IN AROMATHERAPY, AROMA MEDICINE AND AROMATOLOGY

(71) Applicant: Doris HEFTI, Rotkreuz (CH)

(72) Inventor: Doris HEFTI, Rotkreuz (CH)

(21) Appl. No.: 14/551,479

(22) Filed: Nov. 24, 2014

(30) Foreign Application Priority Data
Nov. 29, 2013 (EP) 13 005 575.9

(51) Int. Cl. A61K 36/15; A61K 36/537; A61K 36/53; A61K 36/88; A61K 36/328; A61K 36/63; A61K 36/9068; A61K 36/28; A61K 36/61; A61K 36/752; A61K 36/534; A61K 36/23; A61K 36/185; A61K 36/54; A61K 45/06; A61K 9/00; A61K 36/84

(52) U.S. Cl. CPC A61K 36/15; A61K 36/84; A61K 36/537; A61K 36/53; A61K 36/88; A61K 36/328; A61K 36/63; A61K 36/9068; A61K 36/28; A61K 36/61; A61K 36/752; A61K 36/534; A61K 36/23; A61K 36/185; A61K 36/54; A61K 45/06; A61K 9/0043

(57) ABSTRACT

The present invention refers to a composition for aromatherapy, as well as to its use as a medicine and in aromatherapy for the treatment of troublesome emotions. The composition includes 0.01 to 0.125 vol-% of an active composition and 0.1 to 99.99 vol-% of a transport composition and optionally a carrier oil and/or an alcohol. The active composition contains a mixture of 80 to 120 different essential oils and an alcohol in a volume ratio of 1:500 to 1:5,000 of oils:alcohol. The transport composition is made of one or more essential oils. The treatment involves smelling of the composition simultaneously with a troublesome emotion. In this way, the emotion is sustainably relieved.

The present invention refers to a composition comprising essential oils, which is especially suited for use in aromatherapy and/or as medicine and/or in cosmetics.

Essential oils are extracts soluble in organic solvents or the organic phase from a water vapor distillation of plants or parts of plants, which have a strong odor characteristic for the plant of origin. Essential oils contain secondary plant ingredients, which may in the plant serve for, e.g., attracting insects for pollination, for keeping away vermin or for protection against diseases that are caused by, e.g., bacteria or fungi.

Essential oils mainly consist of mixtures of different terpenes, sesquiterpenes or aromatic compounds. Thus, they comprise many different chemical compounds. Essential oils are liposoluble but do not contain any lipids. In contrast to fatty oils, essential oils evaporate without residue.

Essential oils are formed in oil glands of plants and are stored in plant tissue. They may be located in blossoms, leafs, seeds, fruit peels, roots, resins, barks or wood. Some plants produce essential oils in different parts of the plant, which may differ considerably in their chemical composition, for instance oil from leaf and bark of cinnamon tree.

The most used method for obtaining essential oils from plants is water vapor distillation. The oils of some flowers, such as jasmine, tuberose or mimosa, cannot be obtained by means of water vapor distillation and are, thus, often isolated by extraction. Another known method is cold pressing, which is only used for citrus oils. The rather costly extraction using lipids, the so-called “enfleurage”, is nowadays only performed very rarely.

The term “essential oil” is not protected and is in trade also used for purely synthetic products. In order to distinguish between different types of essential oils, the terms “natural finish”, “natural”, “nature-identical” and “artificial” are used:
Natural finish oils are directly obtained from plants.
Natural oils consist of several natural finish components and are thus also purely obtained from the name-giving plant. Natural oils may not contain any synthetic additives. A mixture of a natural finish oily with synthetic additives is termed natural/nature-identical (N/NI).
The components of nature-identical oils are synthesized based on the chemical composition of natural essential oils, such that the smell similar to the natural oils. The composition of nature-identical oils is soften less complex than the natural basis; for example, nature-identica rosemary oil consists of about eleven components, wheras the natural finish essential oil has about 150 ingredients.
Artificial oils to not have a natural counterpart and are specifically designed for certain smell characteristics.

Essential oils are used differently depending on their characteristics. Often, the use as a fragrance in cosmetics and perfume industry is most common, but some essential oils may also be used for medicine or as technical solvents.

Essential oils are subject to pharmaceutical law, in so far as they are contained in pharmaceutical products. They are described in the German Pharmacopoeia (DAB) as well as in Ph. Eur. (European Pharmacopoeia) and are contained in more than 2000 pharmaceutical products. Being part of cosmetic compositions, essential oils are subject to the rules of the European Cosmetic Directive. In food and consumer products they are subject to the Foodstuffs and consumer products Act. Essential oils are generally freely marketed and may be applied by anybody.

Essential oils have a central role in the natural healing principles of aromatherapy, a special form of phytotherapy for holistic oriented treatment of emotional disorders and diseases, for prophylaxis and for maintenance of good health by means of fragrances.

The term “aromatherapy” refers to the use of essential oils as aroma medicine for improving one's well-being. In Germany, aromatherapy may only be performed by physicians and healing practitioners having an additional qualification, as also essential oils may have undesirable side effects or be toxic. Also, allergic reactions are possible.

Fragrances in general can influence the human body in different ways:
The sense of smell is activated; this leads to a sensation with all the connected side effects (emotional impression, memory, reflex influence on various body functions, etc.).
After ingestion or inhalation, essential oils may also have a direct influence on the organs.
Some essential oils have antibiotic properties, making them suitable for prophylaxis and the treatment of minor infections.
It is also possible to apply oils directly or in diluted form to the skin.

Perception of a smell happens in the olfactory mucosa (Regio olfactoria, also olfactory epithelium), an area of about 2×2.5 cm² of the upper nasal concha and the upper part of the nasal septum. The olfactory epithelium consists of three types of cells, the sensory, support and basal cells. A human has between 10 and 100 million olfactory sensors, which are termed olfactory cells and are embedded in the olfactory epithelium. These olfactory cells re bipolar nerve cells having short dendritic extensions ending in the olfactory mucosa, whereas their long axonal extensions are pointing centrally into the Bulbus olfactorius.

Scent molecules reach the olfactory mucosa with breathing air. In the membrane of the olfactory cells, there is one out of 350 different receptors. If a scent molecule interacts with a receptor, the olfactory cell will send a signal to one of the button-like glomeruli in the olfactory bulb. The olfactory bulb in turn sends the signal to the olfactory cortex, where the stimulus is further analyzed.

The human olfactory system possesses a special electric amplification mechanism that enables olfactory cells of the nose to respond even to extremely weak stimuli. Scientists have established that the sensory cilia of the olfactory cells prepare themselves for the job in a special way: A protein complex pumps chloride ions into the interior of the sensory cilia, thus making them into well-filled chloride stores. When an olfactory stimulus is registered, the weak response of the olfactory receptors immediately opens all the chloride channels located in their external membranes. The release of negatively charged chloride ions causes a loading inversion in the olfactory cell. This in its turn produces strong electric signals that are conveyed to the brain with the olfactory information.

On the one hand, the stimulus is now conveyed to the orbitofrontal cortex of the cerebrum, where it is combined with other impressions, such as taste, tactile or auditory sense or pain, to form an overall impression. Presumably this is the point where the perception enters awareness. On the other hand, also hypothalamus and amygdala are informed of the perceived smell. The amygdala connects smells with emotions and thereby helps evaluating the smell. The hypothalamus regulates the hormonal system. For this reason, it is believe that the effect of pheromones goes via this nerve pathway.

Conveyance to the limbic system, which are effected by only two synapses between olfactory epithelium and amygdala, explain the close connection between fragrances and memory processes and thus the emotional component of fragrance perception. Thereby, it is possible the forgotten memories are recalled to upon perception of certain fragrances.

For the triggering of emotional reactions is the right side of the brain responsible. This side is addressed more intensely by non-verbal stimuli than by verbal messages. Non-verbal stimuli include, among others, colors, images, music, and fragrances. The interaction of different environmental factors is decisive. Isolated effects, so-called single stimuli, have a much more intense effect on the sensory organs than combinations and bundling of stimuli.

Therefore, the fragrance molecule generates an electric signal in the olfactory epithelium, which is conveyed to the nerve cells—the synapse or the synaptic cleft, to be more exact.

Nerve cells exchange information by means of these special contact points, the synapses. New synapses are formed constantly. The brain is similar to a large construction site. Constantly, new extensions are formed on the surfaces of nerve cells. If an extension meets a corresponding structure from a neighboring cell, the ends of the extensions develop to a synapse. Only these contact points enable the transfer of information from one cell to another. If an existing synapse is inefficient or is no longer necessary, it is degraded.

There are large numbers of neurons in our body. Neurons have a cell body and many ramifications, the dendrites and axons. Axones can become up to 1 m long. All dendrites have an “end-foot”, the synapse.

The cerebrum, where conscious information is processed, comprises already about 14 billion neurons, Each neuron has about 1,000 to 10,000 synapses, which are arranged around the axons and dendrites of the other neurons similar to a fur, always leaving a (synaptic) cleft. The wall of these brain cells is formed by a pass-through variable protein membrane. It separates K⁺, Na⁺, Cl⁻ ions and negatively charged protein ions from each other. In this way, an electric voltage is formed. In the neurons, a constant static potential (electric voltage) of 80 mV between the cell interior and the surrounding tissue fluid is maintained. Upon activation of a neuron, the static potential switches to the action potential, which is about 30 mV in opposing polarity. In the course of this change, the protein membrane changes its permeability (pore size) within fractions of a millisecond. The action potential stays intact for one millisecond and is conveyed by the neurites, dendrites and axons with a speed of 120 m/s. The countless synapses can now transfer the action signal via the synaptic cleft in the form of messenger substances and receptor proteins within fractions of a millisecond to other neurons (synapses). Also the electric impulse, which is triggered by the fragrance molecules, is conveyed as an action potential to the pertaining neurons (synapses) in the brain and processed.

It is believed that, upon occurrence of a troublesome emotion, the synaptic cleft is opened and reacts with an “electric thunderstorm” of negatively charged signals. Thus, the synapse is activated and switches from static to action potential. In the course of this switching, the protein membrane changes its permeability (pore size) within fractions of a millisecond. The action potential stays intact for one millisecond and is conveyed by the neurites, dendrites and axons with a speed of 120 m/s. The countless synapses can now transfer the action signal via the synaptic cleft in the form of messenger substances and receptor proteins within fractions of a millisecond to other neurons (synapses). Also the electric impulse, which is triggered by the fragrance molecules, is conveyed as an action potential to the pertaining neurons (synapses) in the brain and processed.

This reaction is absolutely reasonable and sometimes even crucial in acute situations, for example in a dangerous situation where a fast defense reaction is required. However, sometimes people still remember these shocking experiences in every detail even after a long time and “relive” this situation including all emotional consequences, which can be very stressful.

It is believed that these troublesome emotions and symptoms of stress may be caused by the energy in the nervous system still kept in the body and bound, which had originally been allocated by physiological and instinctive mechanisms in order to enable an attack or flight reaction. The biologic system of a traumatized man is still activated, even if the dangerous situation has passed long ago. The negative information from this troublesome experience is still stored on the synapses. This may lead to a palette of symptoms, even though the original object or cause is no longer perceived consciously.

Based on observations like these, it appears obvious that a shock or trauma cannot simply be processed with one's mind, but also needs to be treated emotionally and physically. If a traumatic experience is no longer in someone's consciousness, it can be activated by means of essential oils (olfactory memory) and therapeutic hypnosis or self-hypnosis and transferred from sub-consciousness to consciousness.

However, an effective and in particular long lasting therapy of such troublesome emotions is so far only possible with long-term therapeutic interventions, if at all.

For this reason, it is a problem of the present invention to provide a simple and quickly applicable means for treatment of troublesome emotions and symptoms of stress.

This problem is solved by the following composition and its uses.

The composition of the present invention is particularly suitable for aromatherapy, aroma medicine and aromatology. The composition comprises an active composition and a transport composition. Optionally, it further comprises optionally a carrier oil and/or an alcohol.

According to the present invention, the active composition is present in a share of 0.01 to 0.125 vol-%, while the transport composition has a share of 0.1 to 99.99 vol-%.

The active composition contains a mixture of 80 to 120 different essential oils and an alcohol in a volume ratio of 1:500 to 1:5,000 of oils:alcohol. Therefore, it contains essential oils that are highly diluted in alcohol. The degree of dilution is so high that a human being is usually not able to smell the essential oils of the active composition.

The transport composition consists of one or more different essential oils. It consists of no more than 35 different essential oils. The concentration of the transport composition is such that a human being is able to smell it.

The carrier oil, if present, is selected from the group consisting of avocado oil, calophyllum oil, hemp oil, St. John's wort oil, jojoba oil, coconut fat, macadamia nut oil, almond oil, evening primrose oil, olive oil, calendula oil, sallow thorn oil, black cumin oil, sesame oil, shea butter, grape seed oil, monk's pepper oil, wild rose oil, and mixtures thereof. The share of carrier oil is no more than 99.98 vol-%.

The alcohol present in the active composition and the additional alcohol optionally present in the composition of the present invention are independently of one another selected from the group consisting of methanol, ethanol, propanol, and isopropanol. Preferably, in both cases, ethanol is contained. The share of alcohol is no more than 50 vol-%.

The composition of the present invention acts anxiolytic, liberating, harmonizing, vitalizing, relieving, grounding, clarifying, relaxing, stress relieving, nervine, consoling, balancing, mood rasing, stabilizing, calming.

In particular, however, it has been found that the composition of the present invention is able to completely relieve troublesome emotions.

For this, the emotional triggers are reactivated, for instance by imagining the troublesome situation, by means of an olfactory stimulus or by hypnosis, such that the troublesome experience is returned from sub-consciousness to consciousness. During this, the person concerned will typically feel strong (troublesome) emotions.

By means of the person concerned smelling the composition of the present invention once he/she feels these emotions, the olfactory molecules of the composition and the electric impulses resulting therefrom reach the synapse chain corresponding to the negative experience and sustainably remove the emotion, which is perceived as negative. Thereafter, these troublesome emotions can no longer be triggered.

It is believed that the positive electric impulse (odor molecules) hits the negative electric impulse (troublesome emotion) at the synapse, whereby the two impulses neutralize each other. Thanks to this, the troublesome emotion is sustainably relieved. Therefore, by recalling the troublesome emotion and the simultaneous smelling of the composition of the present invention, this emotion is fully removed.

Application of the composition of the present invention is very simple and cheap: Simply by smelling the composition, a positive effect is achieved. There are no known undesired side-effects, not even in combination with medicaments. Also, there is no risk of over dosing.

The treatment with the composition of the present invention starts a holistic process of resolution. No symptoms are suppressed in the course of this. Furthermore, the user may choose completely freely when and where which subjects are resolved. Hence, he can also fully decide on the speed of treatment.

It has been found the degree of dilution of the active composition plays a very critical role for this effect. For instance, experiments with a significantly higher concentration of essential oils were not successful. Also, a certain minimal concentration appears to be necessary. The effectiveness of the composition of the present invention on the level of consciousness has been proven by means of positive multicenter observation of progress.

It is believed that the transport composition is necessary for enabling the electric signal of the composition of the present invention to reach the nerve cells and synapses and thus for a signal transmission.

According to a preferred embodiment, the active composition contains essential oils and alcohol in a volume ratio of 1:1,000 to 1:2,000. Preferably, the volume ratio of oils:alcohol is 1:1,300 to 1:1,400, more preferably 1:1,370 bis 1:1,380.

The best results have been achieved with a volume ratio of 1:1,378 of essential oils to alcohol in the active composition. In particular, it has been observed that this led to a sustainable relieve of the troublesome emotions.

According to a preferred embodiment, the active composition comprises a mixture of 90 to 110, preferably of 95 to 100 and more preferably of 98 different essential oils. It has been found that such a mixture allows for a holistic treatment of a large variety of troublesome emotions. In particular, it was possible to resolve traumata, shock, sickness caused by stress, psychological and somatic disorders, tension, anxiety, listlessness, loneliness, emotional pain, exhaustion, nervousness, powerlessness, sadness, excessive demands, restlessness, desperation, and/or anger.

Preferably, the active composition comprises at least one essential oil of each mentally balancing, opening, mentally relaxing, calming, holistically relaxing, physically relaxing, grounding, physically balancing, physically inspiring, vitalizing, holistically inspiring, mentally inspiring, and stimulating character.

Preferably, the essential oils contained in the active composition include monoterpenes, monoterpenoid ketones, monoterpenoid aldehydes, monoterpenoid alcohols, monoterpenoid phenols, monoterpenoxides, monoterpenoid ethers, monoterpenoid esters, sesquiterpenes, sesquiterpenoid ketones, sesquiterpenoid aldehydes, sesquiterpenoid alcohols, sesquiterpenoxides, deterpenoid alcohols, aromatic aldehydes, aromatic ketones, aromatic acids, aromatic esters, aromatic alcohols, cumarins, furocumarins, eugenol, and cinnamaldehyde.

Preferably, the active composition contains exclusively natural essential oils.

According to a preferred embodiment, the essential oils of the active composition are selected from the group consisting of sage (Salvia officinalis); butterfly lavender (Lavandula stoechas L.); valerian (Valeriana officinalis); iris (Iris pallida var. florentina); myrrh (Commiphora myrrha or Commiphora molmol); osmanthus (Osmanthus fragrans); Atlas cedar (Cedrus atlantica); ginger (Zingiber officinalis); chamomile, in particular true blue or true chamomile (Matricaria camomilla or Chamomilla recutita); Manuka (Leptospermum scoparium); balm (Melissa officinalis CT Citral); spikenard (Nardostachys jatamansi); patchouli (Pogostemon cablin); yarrow (Achillea millefolium L.); vetiver (Vetiveria zizanoides); Virginia juniper (Juniperus virginiana L.); ylang-ylang (Cananga odorata var. genuina); carrot seed (Daucus carotta L.); sandalwood (Santalum album or Santalum austrocaledonicum); basil Ct Linalool (Ocimum basilicum); hierbabuena (Mentha citrata L.); geranium (Pelargonium graveolens); coriander seed (Coriandrum sativum L.); linaloe (Bursera delpechiana); neroli (Citrus aurantium var. amara); palmarosa (Cymbopogon martinii); peppermint (Mentha×piperita); Damask Rose (Rosa damascena); rose wood (Aniba parviflora or Aniba rosaeodora); spike lavender (Lavandula spica or Lavandula latifolia); thyme CT Linalool (Thymus vulgaris or Thymus officinalis); thyme CT Thujanol (Thymus vulgaris CT Thujanol-4); cajeput (Melaleuca cajeputi or Melaleuca leucadendron); eucalyptus globulus (Eucalyptus globulus); eucalyptus radiata (Eucalyptus radiata); cardamom (Elettaria cardamomum L.); laurel (Laurus nobilis L.); niaouli (Melaleuca viridiflora or Melaleuca quinquinervia); ravintsara (Cinnamomum camphora); resemary CT Cineol (Rosmarinus officinalis); thyme mastichina (Thymus mastichina); ysop decumbens (Hyssopus officinalis var. decumbens); verbena (Lippia citriodora Kuntze); lemongrass (Cymbopogon flexuosus); litsea (Litsea cubeba); lemon eucalyptus (Eucalyptus citriodoria); angelica (Angelica archangelica); bergamot (Citrus aurantium var. bergamia); cistus (Cistus ladaniferus L.); Douglas fir (Pseudotsuga menziesii); elemi (Canarium luzonicum A.); tarragon (Artemisia dracunculus L.); Siberian spruce (Abies sibirica); galbanum (Ferula galbaniflua); grapefruit (Citrus paradisi); pine (Pinus sylvestris); cumin (Cuminum cyminum L.); mountain pine (Pinus mugo); lime (Citrus aurantifolia); marjoram (Origanum majorana or Majorana hortensis); tangerine (Citrus madurensis or Citrus reticulata); Amdean myrtle (Myrtus communis); morocco myrtle (Myrtus communis CT Myrtenylacetat); orange (Citrus sinensis); black pepper (Piper nigrum); grand fir (Abies grandis L.); rosemary CT Verbenon (Rosmarinus officinalis); tea tree (Melaleuca alternifolia); juniper berry (Juniperus communis); olibanum (Boswellia Carterii); citron (Citrus medica); lemon (Citrus limon(um)); cypress (Cupressus sempervirens); thyme CT Thymol (Thymus vulgaris or Thymus officinalis); cassia (Cinnamomum cassia or Cinnamonum aromaticum); cloves (Syzygium aromaticum or Eugenia caryophyllus); tulsi (Ocimum sanctum L.); cinnamon leaf (Cinnamomum ceylanicum); aniseed (Pimpinella anisum); fennel, in particular sweet fennel (Foeniculum vulgare var. dulce); tonka (Dipteryx odorata); immortelle (Helichrysum italicum G.); chamomile, in particular Roman chamomile (Anthemis nobilis or Chamaemelum nobile); lavandin (Lavandula hybrida); lavender (Lavandula officinalis or Lavandula vera or Lavandula angustifolia); muscatel sage (Salvia sclarea); petit-grain bitter orange (Citrus aurantium L. var. pumilia); Eritrean incense (Boswellia carterii); sugar birch (Betula lenta); vanilla (Vanilla planifolia); Siamese benzoe (Styrax tonkinensis or Styrax benzoin); champaca (Michelia champaca L.); heather (Spartium junceum L.); sambac jasmine (Jasminum sambac); petit-grain mandarin (Citrus reticulata blanco); wintergreen (Gaultheria fragrantissima); and ylang-ylang (Cananga odorata var. genuina).

Particularly preferably, the active composition comprises all or essentially all of the before-mentioned essential oils.

According to a preferred embodiment, the composition of the present invention comprises 0.04 to 0.07 vol-% of the active composition, particularly preferably 0.06 vol-%.

In principle, the transport composition may consist of a single essential oil.

Preferably, however, the transport composition consists of a mixture of 7 to 35 different essential oils, more preferably of 14 to 28 different essential oils, and in particular of 21 different essential oils. Preferably, the essential oils are contained in equal volume shares, but also other ratios are possible.

According to a preferred embodiment, the essential oils of the transport composition are selected from the group consisting of Vetiveria zizanoides, Daucus carotta L., Nardostachys jatamansi, Santalum album, Santalum austrocaledonicum, Cananga odorata var. genuina, Elettaria cardamomum L., Citrus paradisi, Cymbopogon flexuosus, Litsea Cubeba, Pelargonium graveolens, Melissa officinalis CT Citral, Rosa damascena, Commiphora myrrha, Commiphora molmol, Salvia officinalis, Melaleuca cajeputi, Melaleuca leucadendron, Hyssopus officinalis var. decumbens, Canarium luzonicum A., Ferula galbaniflua, Boswellia Carterii, Cedrus atlantica, and Citrus limonum.

According to a particularly preferred embodiment, the transport composition consists of essential oils from vetiver (Vetiveria zizanoides), carrot seed (Daucus carotta), spikenard (Nardostachys jatamansi), sandal wood (Santalum album), complete ylang-ylang (Cananga odorata), cardamom (Elettaria cardamomum), grapefruit (Citrus paradisi), lemongrass (Cymbopogon flexuosus), litsea cubeba (Litsea cubeba), geranium (Pelargonium graveolens), balm (Melissa officinalis), rose (Rosa damascena), myrrh (Commiphora myrrha), sage (Salvia officinalis), cajeput (Melaleuca cajeputi), ysop (Hyssopus officinalis), elemi (Canarium luzonicum), galbanum (Ferula galbaniflua), incense (Boswellia carterii), Atlas cedar (Cedrus atlantica), and lemon (Citrus limon). Preferably, all these oils are present in equal shares.

The above-mentioned 21 essential oils usually comprise:

| | |
|---|---|
| Vetiver | 45-50% vetivene, 35% vetiverol/khusimol, 15% |
| | vetivone/vetiveron/khusimon |
| Carrot | 50-60% carotol caucol, 15-25% pinene, 10-20% |
| seed | β-bisabolen, 2-5% linaool, 3% geranyl acetate |
| Spikenard | 60-66% patchoulene/gurjunen, 8-15% valeranon/ |
| | β-jonon, 6-8% patchouli alcohol/valerianol |
| Sandal | 85-95% santalole, 5-10% santalene |
| wood | |
| Ylang- | 55-70% germacren/β-caryophyl, 10-20% benzyl |
| ylang | benzoate/benzyl acetate, 10-20% limalool, 7- |
| | 15% p-cresol methylether, 12% geranyl acetate |
| Cardamom | 35-50% 1,8-cineol, 32-45% terpinyl acetate |
| Grapefruit | 90-98% (1)-limonene (mercaptan in traces), |
| | 0.5-1.8% nootkaton |
| Lemongrass | 70-85% citral, 5-10% limonene, up to 10% |
| | farnesol, up to 6% geraniol, 3% farnesal |
| Litsea | 70-80% citral, 10-15% limonen, 5-10% linalool/ |
| Cubeba | geraniol/nerol, up to 4.5% methylheptenone |
| Geranium | 50-65% citronellol/geraniol, 15-30% geranyl |
| | acetate, 5-10% iso-menthon, 5% citral, 3-5% |
| | rose oxide |
| Balm | 40-60% β-caryophyllene (up to 30%), 25-55% |
| | citral |
| Rose | 65-75% citronellol/geraniol, up to 4% |
| | citronellyl acetate/geranyl acetate, 2-3% |
| | phenylethylalcohol, 2-3% methyleugenol, up to |
| | 1% rose oxide, up to 1% rose ketone |
| Myrrh | Up to 60% furanosesquiterpene, 20-45% |
| | elemene/copaene |
| Sage | 30-60% thuyon, 8-15% 1,8-cineol, 5-15% pinene, |
| | 5-10% borneol |
| Cajeput | 50-65% 1,8-cineol, 25-40% pinene, 6-15% α- |
| | terpineol |
| Ysop | 40-60% 1,8-cineol, 20-30% β-pinene, 6% |
| | isopinocamphone |
| Elemi | 70-80% limonene (up to 55%), 15-17% elemol (up |
| | to 16%), 3-6% elemicin |
| Galbanum | 45.1-58.8% β-pinene, 2.0-12.1% 3-caren, 5.7- |
| | 12.0% α-pinene, up to 6.4% sabinene, up to 4.6% |
| | β-myrcene, 2.7-4.0% (+)-limonene , up to 2.4% |
| | elemene, 1.6-1.8% 1,3,5-undecatriene, up to 1.2% ocimene |
| Incense | 55% octyl acetate, 8% octanal, 2.5% incensol |
| Atlas | 75-80% himachalene, 3-15% himachalol, 3-12% |
| cedar | atlanton, 1% aimachalene oxide |
| Lemon | 80-95% (+)-limonene (60-80%), 3-10% citral |

Preferably, the transport composition contains exclusively natural essential oils.

According to a preferred embodiment, the composition of the present invention comprises 0.5 to 20 vol-% of the transport composition, preferably 1 to 5 vol-%, more preferably 2.5 to 3 vol-%, and in particular 2.94 vol-%.

Small ratios of the transport composition are particularly advantageous in so far as compositions comprising no more than 3% of essential oils are considered as cosmetics. This means that they do not fall under the Therapeutic Products Act and a market authorization is not necessary. In addition, also the odor intensity of the composition of the present invention is somewhat reduced.

Particularly preferably, the composition of the present invention hence comprises 0.06 vol-% of the active composition and 2.94 vol-% of the transport composition. The remaining shares are preferably formed by a carrier oil, whereby also alcohol or other components may be contained.

According to a preferred embodiment, the active composition and the transport composition contain exclusively natural essential oils, particularly preferable natural essential oils from biologic production.

According to a preferred embodiment, the composition of the present invention comprises at least 50 vol-% of the carrier oil, preferably at least 80 vol-%, more preferably at least 95 vol-%, in particular at least 97 vol-%, wherein the share of the carrier oil is not more than 99.89 vol-%. Thanks to this, the composition of the present invention will be considered as a cosmetic.

Alternatively, it is also possible that the composition of the present comprises less or no carrier oil. In particular, the composition may also be intended for being diluted prior to application, for instance with a carrier oil.

In a further aspect, the present invention also relates to the use of the composition of the present invention as medicine.

In particular, the composition of the present invention is suitable for the treatment of emotional disorders and for promoting general well-being.

The composition is especially well suited for the treatment of traumata, shock, sickness caused by stress, psychological and somatic disorders, tension, anxiety, listlessness, loneliness, emotional pain, exhaustion, nervousness, powerlessness, sadness, excessive demands, restlessness, desperation, and/or anger.

In a further aspect, the present invention also relates to the use of the composition of the present invention in aromatherapy. It has in particular been proven to be helpful in the treatment of traumata, shock, sickness caused by stress, psychological and somatic disorders, tension, anxiety, listlessness, loneliness, emotional pain, exhaustion, nervousness, powerlessness, sadness, excessive demands, restlessness, desperation, and/or anger.

The present invention is further illustrated by means of the following examples, which are not to be construed as limiting:

### Example of a Composition of the Present Invention

0.06 vol-% active composition

2.94 vol-% transport composition

97.00 vol-% carrier oil

Active Composition:

98 essential oils in equal shares from sage (Salvia officinalis); butterfly lavender (Lavandula stoechas L.); valerian (Valeriana officinalis); iris (Iris pallida var. florentina); myrrh (Commiphora myrrha or Commiphora molmol); osmanthus (Osmanthus fragrans); Atlas cedar (Cedrus atlantica); ginger (Zingiber officinalis); chamomile, in particular true blue or true chamomile (Matricaria camomilla or Chamomilla recutita); Manuka (Leptospermum scoparium); balm (Melissa officinalis CT Citral); spikenard (Nardostachys jatamansi); patchouli (Pogostemon cablin); yarrow (Achillea millefolium L.); vetiver (Vetiveria zizanoides); Virginia juniper (Juniperus virginiana L.); ylang-ylang (Cananga odorata var. genuina); carrot seed (Daucus carotta L.); sandalwood (Santalum album or Santalum austrocaledonicum); basil Ct Linalool (Ocimum basilicum); hierbabuena (Mentha citrata L.); geranium (Pelargonium graveolens); coriander seed (Coriandrum sativum L.); linaloe (Bursera delpechiana); neroli (Citrus aurantium var. amara); palmarosa (Cymbopogon martinii); peppermint (Mentha×piperita); Damask Rose (Rosa damascena); rose wood (Aniba parviflora or Aniba rosaeodora); spike lavender (Lavandula spica or Lavandula latifolia); thyme CT Linalool (Thymus vulgaris or Thymus officinalis); thyme CT Thujanol (Thymus vulgaris CT Thujanol-4); cajeput. (Melaleuca cajeputi or Melaleuca leucadendron); eucalyptus globulus (Eucalyptus globulus); eucalyptus radiata (Eucalyptus radiata); cardamom (Elettaria cardamomum L.); laurel (Laurus nobilis L.); niaouli (Melaleuca viridiflora or Melaleuca quinquinervia); ravintsara (Cinnamomum camphora); resemary CT Cineol (Rosmarinus officinalis); thyme mastichina (Thymus mastichina); ysop decumbens (Hyssopus officinalis var. decumbens); verbena (Lippia citriodora Kuntze); lemongrass (Cymbopogon flexuosus); litsea (Litsea cubeba); lemon eucalyptus (Eucalyptus citriodoria); angelica (Angelica archangelica); bergamot (Citrus aurantium var. bergamia); cistus (Cistus ladaniferus L.); Douglas fir (Pseudotsuga menziesii); elemi (Canarium luzonicum A.); tarragon (Artemisia dracunculus L.); Siberian spruce (Abies sibirica); galbanum (Ferula galbaniflua); grapefruit (Citrus paradisi); pine (Pinus sylvestris); cumin (Cuminum cyminum L.); mountain pine (Pinus mugo); lime (Citrus aurantifolia); marjoram (Origanum majorana or Majorana hortensis); tangerine (Citrus madurensis or Citrus reticulata); Amdean myrtle (Myrtus communis); morocco myrtle (Myrtus communis CT Myrtenylacetat); orange (Citrus sinensis); black pepper (Piper nigrum); grand fir (Abies grandis L.); rosemary CT Verbenon (Rosmarinus officinalis); tea tree (Melaleuca alternifolia); juniper berry (Juniperus communis); olibanum (Boswellia Carterii); citron (Citrus medica); lemon (Citrus limon(um)); cypress (Cupressus sempervirens); thyme CT Thymol (Thymus vulgaris or Thymus officinalis); cassia (Cinnamomum cassia or Cinnamonum aromaticum); cloves (Syzygium aromaticum or Eugenia caryophyllus); tulsi (Ocimum sanctum L.); cinnamon leaf (Cinnamomum ceylanicum); aniseed (Pimpinella anisum); fennel, in particular sweet fennel (Foeniculum vulgare var. dulce); tonka (Dipteryx odorata); immortelle (Helichrysum italicum G.); chamomile, in particular Roman chamomile (Anthemis nobilis or Chamaemelum nobile); lavandin (Lavandula hybrida); lavender (Lavandula officinalis or Lavandula vera or Lavandula angustifolia); muscatel sage (Salvia sclarea); petit-grain bitter orange (Citrus aurantium L. var. pumilia); Eritrean incense (Boswellia carterii); sugar birch (Betula lenta); vanilla (Vanilla planifolia); Siamese benzoe (Styrax tonkinensis or Styrax benzoin); champaca (Michelia champaca L.); heather (Spartium junceum L.); sambac jasmine (Jasminum sambac); petit-grain mandarin (Citrus reticulata blanco); wintergreen (Gaultheria fragrantissima); and ylang-ylang (Cananga odorata var. genuina); diluted with 96% ethanol (potabile) in a ratio of essential oils to ethanol of 1:1,378.

Transport Composition:

21 essential oils in equal shares from vetiver (Vetiveria zizanoides), carrot seed (Daucus carotta), spikenard (Nardostachys jatamansi), sandal wood (Santalum album), complete ylang-ylang (Cananga odorata), cardamom (Elettaria cardamomum), grapefruit (Citrus paradisi), lemongrass (Cymbopogon flexuosus), litsea cubeba (Litsea cubeba), geranium (Pelargonium graveolens), balm (Melissa officinalis), rose (Rosa damascena), myrrh (Commiphora myrrha), sage (Salvia officinalis), cajeput (Melaleuca cajeputi), ysop (Hyssopus of ficinalis), elemi (Canarium luzonicum), galbanum (Ferula galbaniflua), incense (Boswellia carterii), Atlas cedar (Cedrus atlantica), and lemon (Citrus limon).

### Multicenter Observation of Progress in the Consciousness Stadium Using the Composition of the Present Invention

Instructed by therapists, medical doctors and psychologists.

Period: 17 Mar. 2013-26 Nov. 2013

Evaluation of 305 data collections (state November 2013) on a scale of 0 to 10 (0=no emotion; 10=extremely strong emotion)

| | | Personal | Of which | Of which | Anger | | Restlessness | | Nervousness | | Listlessness | | Sadness | | Loneliness | |
|---|---|---|---|---|---|---|---|---|---|---|---|---|---|---|---|---|
| Age | Anonymous | data known | femal | male | V | N | V | N | V | N | V | N | V | N | V | N |
| 15-25 | 37 | 48 | 69 | 16 | 8.3 | 1.5 | 6.9 | 0.5 | 6.7 | 0.3 | 4.5 | 1.5 | 9.0 | 0.3 | 8.9 | 0.3 |
| 26-45 | 12 | 72 | 59 | 25 | 7.5 | 0.5 | 7.2 | 0.7 | 7.8 | 0.8 | 6.8 | 1.2 | 8.9 | 0.5 | 9.5 | 2.1 |
| 46-65 | 13 | 63 | 54 | 22 | 8.9 | 0.4 | 4.5 | 0 | 7.6 | 1.2 | 7.2 | 2.4 | 7.8 | 0.8 | 7.9 | 1.8 |
| 66-85 | 45 | 15 | 49 | 11 | 6.5 | 1.7 | 5.6 | 0.2 | 5.3 | 0.8 | 0 | 0 | 6.3 | 1.6 | 9.5 | 2.6 |

| | | | | | | | | | | | | | Excessive | | | |
|---|---|---|---|---|---|---|---|---|---|---|---|---|---|---|---|---|
| | | Personal | Of which | Of which | Exhaustion | | Powerlessness | | Anxiety | | Imbalance | | demands | | Somatic pain | |
| Age | Anonymous | data known | femal | male | V | N | V | N | V | N | V | N | V | N | V | N |
| 15-25 | 37 | 48 | 69 | 16 | 0 | 0 | 0 | 0 | 9.0 | 0.2 | 0 | 0 | 8.9 | 0.1 | 7.9 | 2.5 |
| 26-45 | 12 | 72 | 59 | 25 | 8.6 | 2.4 | 7.9 | 1.5 | 8.5 | 0.5 | 6.7 | 1.5 | 9.8 | 0.5 | 8.3 | 2.7 |
| 46-65 | 13 | 63 | 54 | 22 | 7.3 | 1.9 | 6.8 | 0.3 | 9.3 | 0.8 | 5.4 | 1.0 | 8.6 | 0.7 | 7.2 | 3.1 |
| 66-85 | 45 | 15 | 49 | 11 | 5.8 | 1.5 | 0 | 0 | 7.5 | 1.0 | 0 | 0 | 7.3 | 0.8 | 0 | 0 |

The above tables display mean values; V=before smelling; N=after smelling

Evaluations up to date show a significant reduction of troublesome emotions after intensive smelling of the composition of the present invention within one minute. Best results were achieved for anger, anxiety, and sadness. Also, it has been found that the troublesome emotion connected to an initial experience can often no longer be recalled after smelling the composition of the present invention, not even at a later point in time.

1. A composition for aromatherapy, comprising 0.01 to 0.125 vol-% of an active composition and 0.1 to 99.99 vol-% of a transport composition and optionally a carrier oil and/or an alcohol, wherein
the active composition contains a mixture of 80 to 120 different essential oils and an alcohol in a volume ratio of 1:500 to 1:5,000 of oils:alcohol,
the transport composition consists of one or more, but no more than 35 different essential oils,
the carrier oil is selected from the group consisting of avocado oil, calophyllum oil, hemp oil, St. John's wort oil, jojoba oil, coconut fat, macadamia nut oil, almond oil, evening primrose oil, olive oil, calendula oil, sallow thorn oil, black cumin oil, sesame oil, shea butter, grape seed oil, monk's pepper oil, wild rose oil, and mixtures thereof, and
the alcohol is selected from the group consisting of methanol, ethanol, propanol, and isopropanol, preferably ethanol.

2. The composition according to claim 1, wherein the active composition contains essential oils and alcohol in a volume ratio of 1:1,000 to 1:2,000, preferably in a volume ratio of 1:1,300 to 1:1,400, more preferably in a volume ratio of 1:1,370 bis 1:1,380, and in particular in a volume ratio of 1:1,378.

3. The composition according to claim 1, wherein the active composition comprises a mixture of 90 to 110, preferably of 95 to 100 and more preferably of 98 different essential oils.

4. The composition according to claim 1, wherein the essential oils of the active composition are selected from the group consisting of Salvia officinalis, Lavandula stoechas L., Valeriana officinalis, Iris pallida var. florentina, Commiphora myrrha, Commiphora molmol, Osmanthus fragrans, Cedrus atlantica, Zingiber officinalis, Matricaria camomilla, Chamomilla recutita, Leptospermum scoparium, Melissa officinalis CT Citral, Nardostachys jatamansi, Pogostemon cablin, Achillea millefolium L., Vetiveria zizanoides, Juniperus virginiana L., Cananga odorata var. genuina, Daucus carotta L., Santalum album, Santalum austrocaledonicum, Ocimum basilicum, Mentha citrata L., Pelargonium graveolens, Coriandrum sativum L., Bursera delpechiana, Citrus aurantium var. amara, Cymbopogon martinii, Mentha×piperita, Rosa damascena, Aniba parviflora, Aniba rosaeodora, Lavandula spica, Lavandula latifolia, Thymus vulgaris, Thymus officinalis, Thymus vulgaris CT Thujanol-4, Melaleuca cajeputi, Melaleuca leucadendron, Eucalyptus globulus, Eucalyptus radiata, Elettaria cardamomum L., Laurus nobilis L., Melaleuca viridiflora, Melaleuca quinquinervia, Cinnamomum camphora, Rosmarinus officinalis, Thymus mastichina, Hyssopus officinalis var. decumbens, Lippia citriodora Kuntze, Cymbopogon flexuosus, Litsea cubeba, Eucalyptus citriodoria, Angelica archangelica, Citrus aurantium var. bergamia, Cistus ladaniferus L., Pseudotsuga menziesii, Canarium luzonicum A., Artemisia dracunculus L., Abies sibirica, Ferula galbaniflua, Citrus paradisi, Pinus sylvestris, Cuminum cyminum L., Pinus mugo, Citrus aurantifolia, Origanum majorana, Majorana hortensis, Citrus madurensis, Citrus reticulata, Myrtus communis, Myrtus communis CT Myrtenylacetat, Citrus sinensis, Piper nigrum, Abies grandis L., Rosmarinus officinalis, Melaleuca alternifolia, Juniperus communis, Boswellia Carterii, Citrus medica, Citrus limonum, Cupressus sempervirens, Thymus vulgaris, Thymus officinalis, Cinnamomum cassia, Cinnamonum aromaticum, Syzygium aromaticum, Eugenia caryophyllus, Ocimum sanctum L., Cinnamomum ceylanicum, Pimpinella anisum, Foeniculum vulgare var. dulce, Dipteryx odorata, Helichrysum italicum G., Anthemis nobilis, Chamaemelum nobile, Lavandula hybrida, Lavandula officinalis, Lavandula vera, Lavandula angustifolia, Salvia sclarea, Citrus aurantium L. var. pumilia, Boswellia carterii, Betula lenta, Vanilla planifolia, Styrax tonkinensis, Styrax benzoin, Michelia champaca L., Spartium junceum L., Jasminum sambac, Citrus reticulata blanco, Gaultheria fragrantissima, and Cananga odorata var. genuina.

5. The composition according to claim 1, comprising 0.04 to 0.07 vol-% of the active composition, preferably 0.06 vol-%.

6. The composition according to claim 1, wherein the transport composition consists of a mixture of 7 to 35 different essential oils, preferably of 14 to 28 different essential oils, and in particular of 21 different essential oils, wherein the essential oils are contained in equal volume shares.

7. The composition according to claim 1, wherein the essential oils of the transport composition are selected from the group consisting of Vetiveria zizanoides, Daucus carotta L., Nardostachys jatamansi, Santalum album, Santalum austrocaledonicum, Cananga odorata var. genuina, Elettaria cardamomum L., Citrus paradisi, Cymbopogon flexuosus, Litsea Cubeba, Pelargonium graveolens, Melissa officinalis CT Citral, Rosa damascena, Commiphora myrrha, Commiphora molmol, Salvia officinalis, Melaleuca cajeputi, Melaleuca leucadendron, Hyssopus officinalis var. decumbens, Canarium luzonicum A., Ferula galbaniflua, Boswellia Carterii, Cedrus atlantica, and Citrus limonum.

8. The composition according to claim 1, comprising 0.5 to 20 vol-% of the transport composition, preferably 1 to 5 vol-%, more preferably 2.5 to 3 vol-%, and in particular 2.94 vol-%.

9. The composition according to claim 1, wherein the active composition and the transport composition contain exclusively natural essential oils.

10. The composition according to claim 1, comprising at least 50 vol-% of the carrier oil, preferably at least 80 vol-%, more preferably at least 95 vol-%, in particular at least 97 vol-%, wherein the share of the carrier oil is not more than 99.89 vol-%.

11. The composition according to claim 1 for use as medicine.

12. The composition according to claim 1 for use in the treatment of emotional disorders, in particular of traumata, shock, sickness caused by stress, psychological and somatic disorders, tension, anxiety, listlessness, loneliness, emotional pain, exhaustion, nervousness, powerlessness, sadness, excessive demands, restlessness, desperation, and/or anger.

13. A method for treatment of emotional disorders, in particular by aromatherapy, by application of the composition according to claim 1.

14. The method according to claim 13, whereby traumata, shock, sickness caused by stress, psychological and somatic disorders, tension, anxiety, listlessness, loneliness, emotional pain, exhaustion, nervousness, powerlessness, sadness, excessive demands, restlessness, desperation, and/or anger are treated.
